(19) United States
(12) Patent Application Publication
(10) Pub. No.: US 2003/0139795 A1
(43) Pub. Date: Jul. 24, 2003

(54) Stent delivery system loading tool

(75) Inventor: Richard J. Olson, Blaine, MN (US)

Correspondence Address: VIDAS, ARRETT & STEINKRAUS, P.A., 6109 BLUE CIRCLE DRIVE, SUITE 2000, MINNETONKA, MN, 55343-9185, US

(73) Assignee: SCIMED LIFE SYSTEMS, INC., Maple Grove, MN

(21) Appl. No.: 10/055,836

(22) Filed: Jan. 23, 2002

(51) Int. Cl. A61F 2/06

(52) U.S. Cl. 623/1.11

(57) ABSTRACT

A removable loading tool for use with a catheter assembly wherein the loading tool comprises a body which defines a substantially hollow chamber. The substantially hollow chamber has a first portion and a second portion. The first portion has a first diameter sized to contain at least a portion of the catheter assembly having a stent mounted thereon. The second portion has a second diameter less than that of the first diameter. The body constructed and arranged to protect the at least a portion of the catheter assembly and the stent from undesired contact or damage during loading of a guide wire into the catheter assembly.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

Medical devices such as stents, stent-grafts, grafts, or vena cava filters and catheters, balloon catheters, and medical balloons for their delivery are utilized in a number of medical procedures and situations, and as such their structure and function are well known.

Catheters for example, may be used in a variety of medical procedures. An example of one potential use for a catheter is in PTCA procedures. In typical PTCA procedures, a guiding catheter is percutaneously introduced into the cardiovascular system of a patient through a vessel and advanced through therein until the distal end thereof is at a desired location in the vasculature. A guide wire and a dilatation catheter having a balloon on the distal end thereof are introduced through the guiding catheter with the guide wire sliding through the dilatation catheter. The guide wire is first advanced out of the guiding catheter into the patient's coronary vasculature and the dilatation catheter is advanced over the previously advanced guide wire until the dilatation balloon is properly positioned across the lesion. Once in position across the lesion, the flexible, expandable, preformed balloon is inflated to a predetermined size with a liquid or gas at, to radially compress the arthrosclerotic plaque of the lesion against the inside of the artery wall and thereby dilate the lumen of the artery. The balloon is then deflated to a small profile so that the dilatation catheter may be withdrawn from the patients vasculature and blood flow resumed through the dilated artery.

In angioplasty procedures of the kind described above, there may be injury to or restenosis of the artery, which either necessitates another angioplasty procedure, a surgical by-pass operation, or some method of repairing or strengthening the area. To strengthen the area and help prevent restenosis, a physician can implant an intravascular prosthesis for maintaining vascular patency, commonly called a stent, inside the artery at the lesion. The stent is expanded to a larger diameter for placement in the vasculature, often by the balloon portion of the catheter. Stents delivered to a restricted coronary artery, expanded to a larger diameter by a balloon catheter, and left in place in the artery at the site of a dilated lesion are shown in U.S. Pat. No. 4,740,207 to Kreamer and U.S. Pat. No. 5,007,926 to Derbyshire, the content of which is incorporated herein by reference. Palmaz et al., 156 Radiology 73 (1985) and U.S. Pat. No. 4,733,665 describe introduction of a stent over a balloon catheter (incorporated herein by reference).

When assembling a catheter for insertion into a body vessel, a guide wire is loaded into a guide wire lumen of the catheter. During the loading process the catheter is manipulated, often by hand. In embodiments where the catheter includes a stent disposed about a portion of the catheter, the manipulation of the catheter may cause the stent to be contacted and potentially damaged. Where the stent includes or is coated with a drug or other material, such contact may disturb the coating and impair the proper delivery thereof. In addition, contacting the stent during the guide wire loading process may compromise the sterile field of the stent. Finally, undesired contact with the stent may be sufficient to disturb the position of the stent on the catheter. Disturbing the position of the stent on the catheter could impair the trackability of the catheter as it is advanced through a body lumen as well as potentially cause an impairment with the delivery of the stent to a target location from the catheter.

As a result, it would be beneficial to provide a tool that protects the region of a catheter having a stent mounted thereon from inadvertent contact during the process of loading the system onto a guide wire or into a catheter.

The entire content of all of the patents listed within the present patent application are incorporated herein by reference.

Without limiting the scope of the invention a brief summary of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

The present invention may be embodied in several forms. In at least one embodiment, the invention is directed to a removable loading tool. The loading tool being a substantially hollow member disposed about at least a portion of a catheter. The loading tool providing a protective covering over a region of the catheter having a stent mounted thereon. In some embodiments of the invention the loading tool can have a hinged structure which allows the substantially hollow member to be opened along a longitudinal seam from a closed to an opened position. In some embodiments of the invention wherein the tool includes a hinge structure and seam, the tool may be equipped with one or more fasteners or clips for securing the tool in the closed position. In some embodiments of the invention the loading tool comprises a necked region which can reduce the likelihood of the tool from being moved distally relative to the catheter. In some embodiments of the invention the necked region may have an outer gripping surface, the outer gripping surface may textured and/or include one or more gripping pads.

Additional details and/or embodiments of the invention are discussed below.

### BRIEF DESCRIPTION OF THE SEVERAL VIEWS OF THE DRAWINGS

A detailed description of the invention is hereafter described with specific reference being made to the drawings in which:

FIG. 1 is a perspective view of an embodiment of the invention;

FIG. 2 is a longitudinal cross-section view of an embodiment of the invention;

FIG. 3 is a longitudinal cross-section view of an embodiment of the invention;

FIG. 4 is a side elevational view of the embodiment of the invention shown in the closed state;

FIG. 5 is a side elevational view of an embodiment of the invention shown in FIG. 4 wherein the tool is shown in the open state; and

FIG. 6 is a longitudinal cross-section view of an embodiment of the invention shown in a perspective environment of use.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

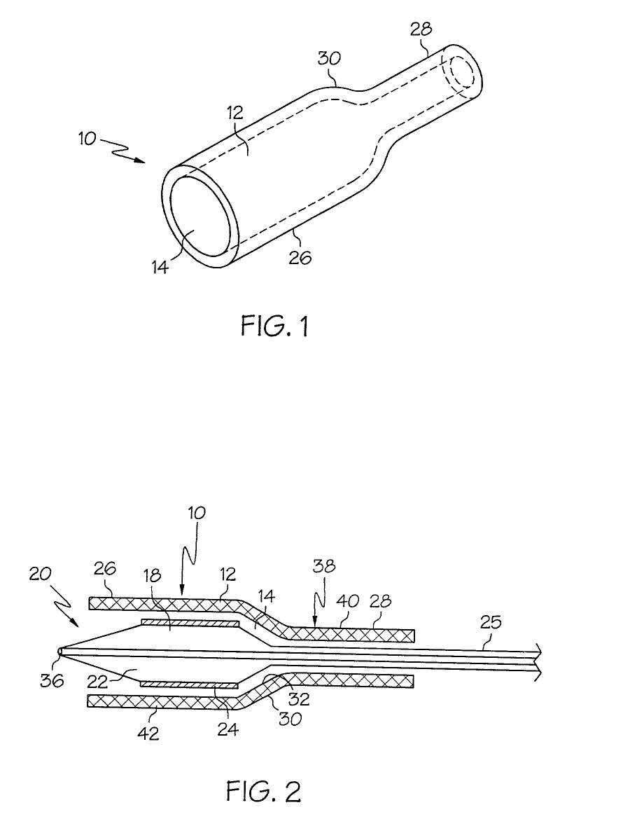

In FIG. 1 a loading tool, indicated generally at 10, is shown. The embodiment of the loading tool 10 shown, comprises a body 12 which defines a substantially hollow chamber or lumen 14. It should be noted, that while in the various embodiments depicted herein body 12 is shown having an elliptical or circular cross-section, the body 12 may be shaped in any manner desired in accordance with the inventive concepts described herein.

Tool 10 may be characterized as having a first or distal portion 26 and a proximal or second portion 28. The tool 10 defines a chamber 14 having a diameter sufficient to allow the body to be placed about a distal region 22 of a catheter assembly 20, such as is shown in FIG. 2. In embodiments where the catheter 20 is configured for stent delivery, the first portion 26 of chamber 14 has a sufficient diameter to allow the stent mounting region 18 of the catheter 20 having a stent 24 thereon to be positioned therein.

The chamber 14 may be sufficiently large to accommodate a bifurcated stent or any other medical device desired for use with catheter 20.

In some embodiments, the first portion 26 of chamber 14 has a diameter of about 0.5 mm to about 5 mm and more preferably a diameter of about 0.7 mm to about 3.8 mm. The proximal portion 28 of the chamber 14 may have a narrower diameter than that of the distal portion 26. The proximal portion 28 of the chamber 14 is sized to accommodate the proximal catheter shaft 25, or the distal portion proximal to the balloon, which is typically a narrower portion of the catheter 20 than stent mounting region 18. Typically, the proximal portion 28 of chamber 14 has a diameter of about 0.2 mm to about 4.9 mm and more preferably about 0.4 mm to about 4.5 mm.

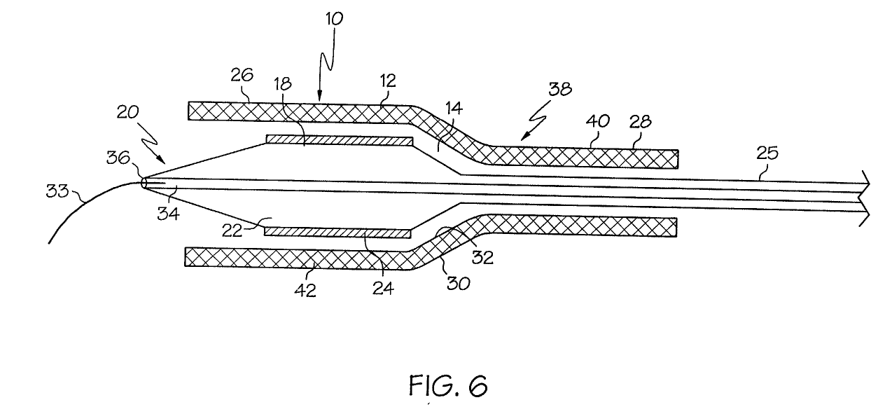

In addition to providing a chamber 14 which shields the stent 24 from external contact, in some embodiments tool 10 includes a neck or necked region 30 which separates the wider distal portion 26 from the narrower proximal portion 28. The neck 30 may be characterized as providing a reduction in diameter between the proximal and distal portions 26 and 28. In some embodiments, the reduction in diameter provided by the neck 30 may be utilized as an engagement surface 32 for engagement of a portion of the stent mounting region 18. By engaging a portion of the stent mounting region 18 the neck 30 prevents the catheter 20 from moving in a proximal direction relative to the tool 10. As a result, when a guide wire 33, such as is shown in FIG. 6, is inserted into the guide wire lumen 34 from the distal end 36 of the catheter 20, the catheter 20 may be held in place as long as the tool 10 is held in place. In those embodiments that include a neck 30, the tool 10 not only protects the portion of the catheter 20 and/or stent 24 contained within the chamber 14 from compression, damage or inadvertent shifting, but may also provide improved stabilization of the catheter 20 within the chamber 14 via the engagement surface 32.

In some embodiments of the invention, tool 10 may also provide a convenient gripping surface 38 which an operator (not shown) may grip and manipulate tool 10 and thus the catheter held therewithin. The gripping surface 38 is preferably positioned on the external surface 40 of the proximal portion 28 of the tool 10, but may be located anywhere on the tool 10. In some embodiments of the invention, the neck 30 or distal portion 26 may be provided with a griping surface 38. Gripping surface 38 may be provided for by providing the external surface 40 of the tool with a texture such as by abrading the surface, or by providing the surface 40 with a coating of a non-lubricious, or even sticky material.

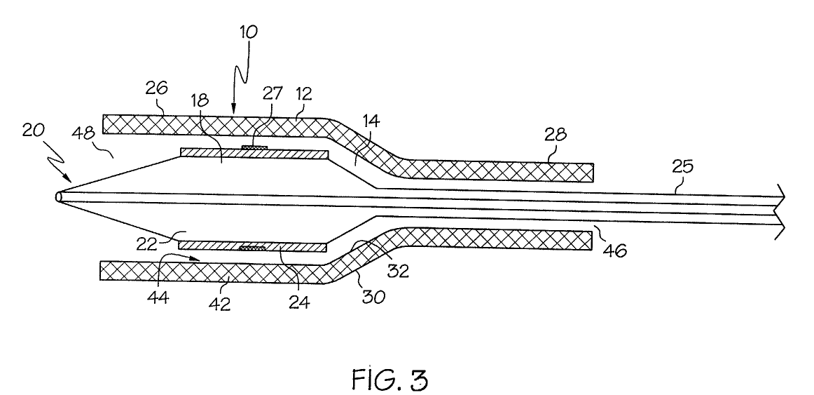

As indicated above, tool 10 is constructed and arranged to prevent or reduce direct contact of the catheter 20 and or stent 24 positioned within the chamber 14 with an operator or other potentially undesired contact source. As such, the tool 10 may be constructed from virtually any material 42 having sufficient structural characteristics to prevent the underlying catheter 20 and/or stent 24 from being compressed or damaged during routine manipulation of the catheter 20 during guide wire insertion. Such materials 42 may include, but are not limited to: metals, polymers, or any other substance etc. Preferably, materials 42 are any materials amenable to sterilization. In at least one embodiment of the invention such as is shown in FIG. 3, the tool 10 may be constructed of a material such as stainless steel and/or polycarbonate, but the chamber 14 has an inner surface 44 of a softer polymer material to prevent damage to the catheter 20 and/or stent 24. The polymer material of inner surface 44 may be any polymer material such as polyethylene. In a preferred embodiment of the invention wherein the stent 24 is a drug delivery stent and includes a coating 27, the inner surface is a layer of inert material relative to the drug or other coating 27 of the stent 24. Preferably, such an inner surface layer is composed of PTFE, HDPE, or any type of urethane or ethylene material.

In those embodiments where stent 24 is a drug delivery stent, at least a portion of the stent 24 protected by chamber 14 includes a coating 27 of a desired substance such as drug, genetic material, cells, a non-genetic therapeutic agent, a polymer matrix having a therapeutic component or any other substance which it would desirable to deliver into a body lumen. In some embodiments the stent may be at least partially coated with one or more of: SIBS (styrene isobutylene styrene); polycarboxylic acids; cellulosic polymers, including cellulose acetate and cellulose nitrate; gelatin, polyvinylpyrrolidone; cross-linked polyvinylpyrrolidone; polyanhydrides including maleic anhydride polymers; polyamides; polyvinyl alcohols; copolymers of vinyl monomers such as EVA; polyvinyl ethers; polyvinyl aromatics; polyethylene oxides; glycosaminoglycans; polysaccharides; polyesters including polyethylene terephthalate; polyacrylamides; polyethers; polyether sulfone; polycarbonate; polyalkylenes including polypropylene, polyethylene and high molecular weight polyethylene; halogenated polyalkylenes including polytetrafluoroethylene; polyurethanes; polyorthoesters; proteins; polypeptides; silicones; siloxane polymers; polylactic acid; polyglycolic acid; polycaprolactone; polyhydroxybutyrate valerate and blends and copolymers thereof; coatings from polymer dispersions such as polyurethane dispersions (BAYHDROL®, etc.); fibrin; collagen and derivatives thereof; polysaccharides such as celluloses, starches, dextrans, alginates and derivatives; hyaluronic acid; squalene emulsions; polyacrylic acid, available as HYDROPLUS® from Boston Scientific Corporation, Natick, Mass., and described in U.S. Pat. No. 5,091,205, the entire contents of which is hereby incorporated herein by reference.

In some embodiments of the invention chamber 14 has a proximal opening 46 and/or a distal opening 48. In embodiments where the tool 10 defines both a proximal opening 46 and a distal opening 48 the catheter 20 may be inserted into chamber 14 by passing the catheter 20 proximally through the openings until the neck 30 prevents further advancement.

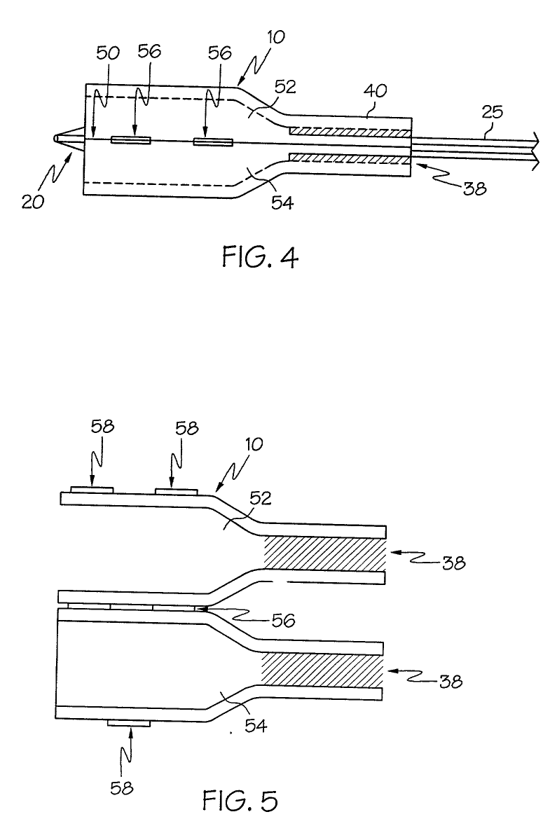

In some embodiments of the invention, an example of which is shown in FIG. 4, it is not necessary to thread the catheter 20 into the chamber 14 as the tool 10 further comprises a longitudinal seam 50 about which two halves 52 and 54 of the tool may be opened or closed such as in a clam-shell relationship. In the embodiment shown, a first half 52 and a second half 54 are joined longitudinally along one side of the seam 50 by one or more hinge components 56. By providing halves 52 and 54 with a hinge components 56 allows the halves 52 and 54 to be capable of moving from the open position shown in FIG. 4 to a closed position such as is shown in FIG. 5 along seam 50 upon application of a predetermined quantity of opening or closing force.

In some embodiments of the invention, once the catheter 20 has been placed within the closed tool 10, such as is shown in FIG. 4, it is desirable to provide the tool 10 with one or more fastening devices 58 to prevent unintended opening of the tool 10. Fastening devices 58 secure the halves 52 and 54 in the closed position by frictional or other type of engagement. The fastening devices 58 may be lockable and may comprise, screws, tabs, clips or other types of fasteners that may be removably secured together. However, in some embodiments, fastening and/or the presence of fastening devices 58 may not be needed as the pressure of an operator's fingers or tools may readily maintain the tool in the closed position.

In addition to being directed to the specific combinations of features claimed below, the invention is also directed to embodiments having other combinations of the dependent features claimed below and other combinations of the features described above.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

1. A removable loading tool for use with a catheter assembly, the loading tool comprising:
a body, the body defining a substantially hollow chamber, the substantially hollow chamber having a first portion and a second portion, the first portion having a first diameter sized to contain at least a portion of the catheter assembly having a stent mounted thereon, the second portion having a second diameter less than that of the first diameter, the body constructed and arranged to protect the at least a portion of the catheter assembly and the stent from undesired contact or damage during loading of a guide wire into the catheter assembly.

2. The loading tool of claim 1 wherein the first portion of the substantially hollow chamber defines a first opening and the second portion of the substantially hollow chamber comprises a second opening, the first opening having a diameter sufficient to allow passage of the catheter assembly having the stent mounted thereon, the second opening having a diameter smaller than the first opening, the diameter of the second opening being sufficient to allow passage of a proximal shaft of the catheter assembly.

3. The loading tool of claim 1 further comprising a neck portion the neck portion connecting the first portion of the substantially hollow chamber to the second portion of the substantially hollow chamber.

4. The loading tool of claim 3 wherein the neck portion defines a neck portion diameter, the neck portion diameter tapering from the first diameter to the second diameter.

5. The loading tool of claim 3 wherein the neck portion is constructed and arranged to engage the at least a portion of a catheter assembly having a stent mounted thereon, thereby preventing the at least a portion of a catheter assembly having a stent mounted thereon from migrating from the first portion of the hollow chamber to the second portion of the hollow chamber.

6. The loading tool of claim 1 wherein the body is constructed from at least one material of the group consisting of: metal, polymer and any combination thereof.

7. The loading tool of claim 1 further comprising an inside surface, the inside surface being immediately adjacent to the body and defining at least the first portion of the substantially hollow chamber, the inside surface comprising a polymer material, the polymer material constructed and arranged to protect the stent from damage during manipulation of the catheter assembly.

8. The loading tool of claim 7 wherein the body is constructed from a different material than the inside surface.

9. The loading tool of claim 7 wherein the body is constructed from a the same material as the inside surface.

10. The loading tool of claim 7 wherein the stent is a drug delivery stent, the polymer material being inert relative to a drug being delivered by the drug delivery stent.

11. The loading tool of claim 7 wherein the polymer material is characterized as being lubricious.

12. The loading tool of claim 1 wherein the body further comprises a gripping surface, the gripping surface comprising at least one surface feature of a portion of an external surface of the body, the gripping surface selected from at least one surface feature of the group consisting of, a textured surface, a surface coating and any combination thereof.

13. The loading tool of claim 1 wherein the body further comprises a first half and a second half, the first half and the second half being separable from one another.

14. The loading tool of claim 13 wherein the first half and the second half are separable along a common seam, the first half being hingedly connected to the second half along at least a portion of one side of the common seam, the first half and the second half being constructed and arranged to hingedly move between an open position and a closed position.

15. The loading tool of claim 13 wherein the first half and the second half define the substantially hollow chamber in the closed position.

16. The loading tool of claim 13 wherein in the open position the first half and the second half are engaged along only one side of the common seam.

17. The loading tool of claim 13 further comprising at least one fastening device, the at least one fastening device, the at least one fastening device removably engaging the first half to the second half in the closed position.

18. The loading tool of claim 16 wherein the at least one fastening device is selected from at least one member of the group consisting of: at least one fastener, at least one screw, at least one tab, at least one clip and any combination thereof.

19. The loading tool of claim 1 wherein the first diameter is about 0.5 mm to about 5 mm.

20. The loading tool of claim 1 wherein the first diameter is about 0.7 mm to about 3.8 mm.

21. The loading tool of claim 1 wherein the second diameter is about 0.2 mm to about 4.9 mm.

22. The loading tool of claim 1 wherein the second diameter is about 0.4 mm to about 4.5 mm.

23. A catheter assembly comprising:
a catheter, the catheter having a catheter body defining a guide wire lumen, a proximal region and a distal region;
a stent, the stent being disposed about at least a portion of the distal region of the catheter body, at least a portion of the stent having a coating,
a removable guide wire loading tool, the tool being disposed about the at least a portion of the distal region of the catheter and at least a portion of the proximal region of the catheter, the tool having a tool body, the tool body defining a substantially hollow chamber.

24. The catheter of claim 23 wherein the substantially hollow chamber comprises a first portion and a second portion, the first portion removably containing the at least a portion of the distal region of the catheter, the second portion removably containing the at least a portion of the proximal region of the catheter.

25. The catheter of claim 24 wherein the tool body is constructed and arranged to protect the stent positioned on the at least a portion of the distal region of the catheter from undesired contact or damage during loading of a guide wire into the guide wire lumen.

26. The catheter of claim 24 wherein the tool body is constructed and arranged to protect the coating from undesired contact or damage during loading of a guide wire into the guide wire lumen.

27. The catheter of claim 23 wherein the coating is selected from at least one member of the group consisting of: drugs, genetic material, cells, a non-genetic therapeutic agent, a polymer matrix having a therapeutic component, and any combination thereof.

28. The catheter of claim 23 wherein the substance is selected from at least one member of the group consisting of: SIBS (styrene isobutylene styrene); polycarboxylic acid; cellulosic polymer, such as cellulose acetate and cellulose nitrate; gelatin, polyvinylpyrrolidone; cross-linked polyvinylpyrrolidone; polyanhydride such as maleic anhydride polymer; polyamide; polyvinyl alcohol; copolymers of vinyl monomers such as EVA; polyvinyl ether; polyvinyl aromatic; polyethylene oxide; glycosaminoglycan; polysaccharide; polyesters such as polyethylene terephthalate; polyacrylamide; polyether; polyether sulfone; polycarbonate; polyalkylenes including polypropylene, polyethylene and high molecular weight polyethylene; halogenated polyalkylenes such as polytetrafluoroethylene; polyurethane; polyorthoester; protein; polypeptide; silicone; siloxane polymer; polylactic acid; polyglycolic acid; polycaprolactone; polyhydroxybutyrate valerate and blends and copolymers thereof; polyurethane dispersions; fibrin; collagen and derivatives thereof; polysaccharides such as celluloses, starches, dextrans, alginates and derivatives; hyaluronic acid; squalene emulsion; polyacrylic acid and any combinations thereof.
